(19) United States
(12) Patent Application Publication
(10) Pub. No.: US 2025/0082457 A1
(43) Pub. Date: Mar. 13, 2025

(54) CONSTRAINT ARRANGEMENT FOR A STENT-GRAFT LOADED ONTO A DELIVERY SYSTEM

(71) Applicant: Cook Medical Technologies LLC, Bloomington, IN (US)

(72) Inventors: Chantelle King, Queensland (AU); Werner Ducke, Queensland (AU)

(73) Assignee: Cook Medical Technologies LLC, Bloomington, IN (US)

(21) Appl. No.: 18/827,361

(22) Filed: Sep. 6, 2024

Related U.S. Application Data
(60) Provisional application No. 63/581,548, filed on Sep. 8, 2023.

(51) Int. Cl. A61F 2/07; A61F 2/95; A61F 2/962

(52) U.S. Cl. CPC A61F 2/07; A61F 2/9517; A61F 2/962; A61F 2002/9511

(57) ABSTRACT

A constraint arrangement for a stent-graft loaded onto a delivery device. The stent-graft has a graft body defining an elongate lumen and a positionable region. At least one apposition release wire releasably secures the graft body to the guide wire cannula along a length of the guide wire cannula. A suture is sewn circumferentially through and around the graft body, such that a plurality of internal suture portions are formed. A constraint loop is formed from a first of the internal suture portions. A plurality of reducing loops are formed from others of the internal suture portions. At least one apposition release wire passes through the constraint loop and each of the plurality of reducing loops, and upon retraction of the apposition release wire releases the constraint loop and each of the reducing loops and, thereby allowing the stent-graft to expand and to release from the guide wire cannula.

### CROSS-REFERENCE TO RELATED APPLICATIONS

This application claims priority from U.S. Provisional Application No. 63/581,548, filed Sep. 8, 2023, which is hereby incorporated by reference herein in its entirety.

### FIELD OF DISCLOSURE

This disclosure relates to medical devices and more particularly to stent-grafts mountable onto a deployment device for endovascular introduction.

### BACKGROUND OF THE DISCLOSURE

This disclosure will be particularly discussed in relation to stent-grafts for placement into the aorta for the treatment of aneurysms. The disclosure, however, is not so restricted and may be applied to stent-grafts for placement in any lumen of the human or animal body.

The accurate positioning of stent-grafts is very important for a number of reasons, including in many cases the need to avoid occlusion of branch arteries. Positioning is complicated however because the diameter of a stent-graft is typically deliberately made larger than the diameter into which it is to be placed to allow for accurate sealing against the vessel wall. Once released from a delivery device, a stent-graft with self-expanding stents will take up a position against the vessel wall.

Many cases of aneurism repair require the accurate placement of stent-graft relative to certain anatomical features such as a fenestration or scallop. Accurate rotational positioning is also important in many cases. Where a stent-graft is to be deployed in a curved part of a vessel, such as the aortic arch, it is also important to for the stent graft to conform to an interior portion of a curved portion of the aorta for instance. It is an object of the disclosure to provide a stent graft system that allows for accurate placement, repositioning and conformance.

Throughout this specification the term distal with respect to a portion of the aorta, a deployment device or a prosthesis is the end of the aorta, deployment device or prosthesis further away in the direction of blood flow away from the heart and the term proximal means the portion of the aorta, deployment device or end of the prosthesis nearer to the heart. When applied to other vessels, similar terms such as caudal and cranial should be understood.

### SUMMARY

A constraint arrangement for a stent-graft loaded onto a delivery device in combination with a stent-graft. The stent-graft has a graft body defining an elongate lumen and a positionable region. At least one apposition release wire releasably secures the graft body to the guide wire cannula along a length of the guide wire cannula. A first suture is sewn circumferentially through and around the graft body, such that a plurality of external suture portions and a plurality of internal suture portions are formed. A constraint loop is formed from a first of the internal suture portions and passes around the guide wire cannula to constrain the positionable region in close apposition to the guide wire cannula. A plurality of reducing loops are formed from others of the internal suture portions, the reducing loops extending radially inwards from the graft body. At least one apposition release wire passes through the constraint loop and each of the plurality of reducing loops, and upon retraction of the apposition release wire releases the constraint loop and each of the reducing loops and, thereby allowing the stent-graft to expand and to release from the guide wire cannula. The apposition release wire may be helical, undulating or spiral. The apposition release wire may weave in and out of the graft. The first suture may be attached at the distal apices of a proximal most internal stent.

The present invention may be practiced according to the claims without some or all of these specific details. For the purpose of clarity, technical material that is known in the technical fields related to the invention has not been described in detail so that the present invention is not unnecessarily obscured.

### BRIEF DESCRIPTION OF THE DRAWINGS

This then generally describes the invention but, to assist with understanding, reference will now be made to the accompanying drawings which show preferred embodiments of the invention.

FIG. 1A is a side view of a stent-graft;

FIG. 1B is a side view of a portion of a stent-graft and delivery device assembly;

FIG. 2A is a stylized end view of inside a proximal end of the stent-graft shown in FIG. 1B;

FIG. 2B is a close-up diagrammatic view of inside a proximal end of the stent-graft shown in FIG. 2A;

FIG. 3 is a similar view to that of FIG. 2B, but shows an alternative suture arrangement;

FIG. 4 is a close-up view of a proximal end portion of the stent-graft and delivery device assembly of FIGS. 1B, 2A and 2B;

FIGS. 5 and 6 are diagrammatic views showing deployment of the stent-graft and delivery device assembly of FIGS. 1B, 2A and 2B with an aortic arch of a patient;

FIG. 7A is a side view of an alternative stent-graft;

FIG. 7B is a side view of an alternative stent-graft and delivery device assembly;

FIG. 8 is a close-up view of a proximal end portion of the stent-graft and delivery device assembly of FIG. 7B;

FIG. 9 is a side view of a handle end of the delivery device of FIG. 8;

FIG. 10A is an isometric view of a stent-graft and delivery device assembly;

FIG. 10B a similar view to that of FIG. 10A, but shows the stent-graft released;

FIG. 11A is a front view of a constraint arrangement for a stent-graft loaded onto a delivery system;

FIG. 11B is a front view of another constraint arrangement for a stent-graft loaded onto a delivery system;

FIG. 12A is a close-up view of FIG. 11A from within a lumen of the stent-graft, showing routing of a suture being tied around a cannula;

FIG. 12B is a similar view to that of FIG. 12A, but with the suture tightened;

FIG. 13 is a further view of the stent-graft shown in FIG. 11A, again from inside the lumen, prior to securement;

FIG. 14 is a close-up view of a portion of the constraint arrangement of FIGS. 11A, 12A, 12B and 13;

FIGS. 15A and 15B are diagrammatic views showing routing of the suture shown in FIG. 11A in a constraining and non-constraining condition respectively;

FIGS. 16A, 16B and 16C are progressive illustrations showing the constraint arrangement of the previous figures being deployed and then released within a lumen of a patient;

FIG. 17 is a front view of an alternative constraint arrangement for a stent-graft loaded onto a delivery system, according to a second aspect of the disclosure;

FIG. 18 is a further view of the stent-graft shown in FIG. 17, from inside the lumen, prior to securement;

FIGS. 19A and 19B are diagrammatic views showing routing of the suture shown in FIG. 17 in a constraining and non-constraining condition respectively;

FIG. 20 is a front view of a constraint arrangement for an alternative stent-graft loaded onto a delivery system;

FIG. 21 is an illustration of a stylized end view of inside a proximal end of a stent-graft, showing two sutures with two release wires;

FIGS. 22 and 23 are illustrations of stylized end view of inside a proximal end of a stent-graft, showing two sutures with a single release wire; and

FIG. 24 is an illustration of a stylized end view of inside a proximal end of a stent-graft, showing one suture with two release wires.

### DESCRIPTION OF PREFERRED EMBODIMENTS

The terms “about,” “substantially,” “generally,” and other terms of degree, when used with reference to any volume, dimension, proportion, or other quantitative or qualitative value, are intended to communicate a definite and identifiable value within the standard parameters that would be understood by one of skill in the art (e.g., equivalent to a mechanical engineer with experience in this field), and should be interpreted to include at least any legal equivalents, minor but functionally-insignificant variants, standard manufacturing tolerances, and including at least mathematically significant figures (although not required to be as broad as the largest range thereof), including a variance of up to, for example 5%, 2%, 1%, or less or more as would be deemed appropriate by one of skill in the art.

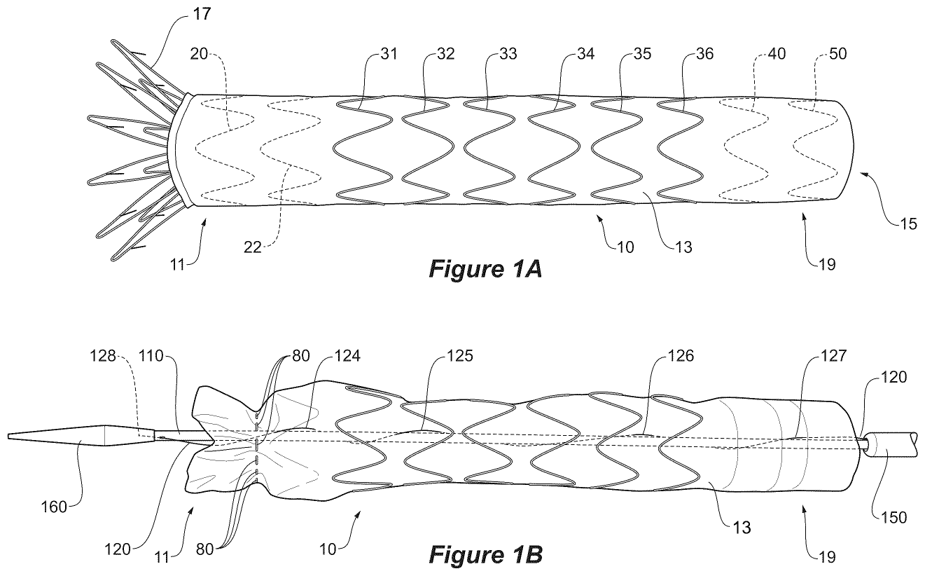

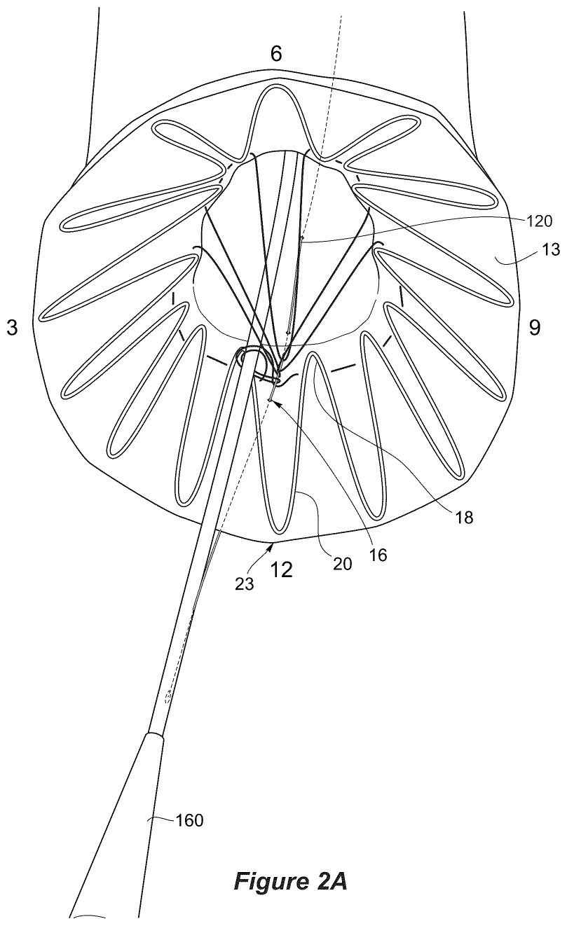

Referring to FIG. 1A, a side view of a stent-graft 10 that could be used with, or form part of, an embodiment of the disclosure is shown. The stent-graft 10 extends between a proximal end portion 11 and a distal end portion 19 and has a graft body 13 defining an elongate lumen 15, and a plurality of stents 17, 20, 22, 31, 32, 33, 34, 35, 36, 40, 50. In the particular embodiment shown, stents 20, 22 at one end of the stent-graft 10 are internal stents, and similarly, the stents 40, 50 at an opposite end of the stent-graft 10 are internal stents, while in between, are external stents 31, 32, 33, 34, 35, 36. The internal stents 20, 22, 40, 50 facilitate the provision of sealing zones at respective ends of the stent-graft 10. It is understood that other suitable stenting arrangements, including, for example, the utilization of external stents at the distal end and/or the proximal end, may be used based on various considerations, including patient anatomy and necessity of fixation.

The stent-graft 10 of FIG. 1A also has an uncovered terminal stent 17 with barbs to prevent migration along an inside of a vessel once deployed. Other stent-grafts without an uncovered terminal stent, such as the stent-graft 10 shown in FIG. 1B, could also be used with embodiments of the disclosure.

FIG. 1B is a side view of a portion of a stent-graft and delivery device assembly. The stent-graft 10 of the assembly is similar to that shown in FIG. 1A, but does not have an uncovered terminal stent 17 of the stent-graft in FIG. 1A. In other embodiments of the stent-graft and delivery device assembly shown in FIG. 1A, an uncovered terminal stent 17, with or without barbs, may be provided.

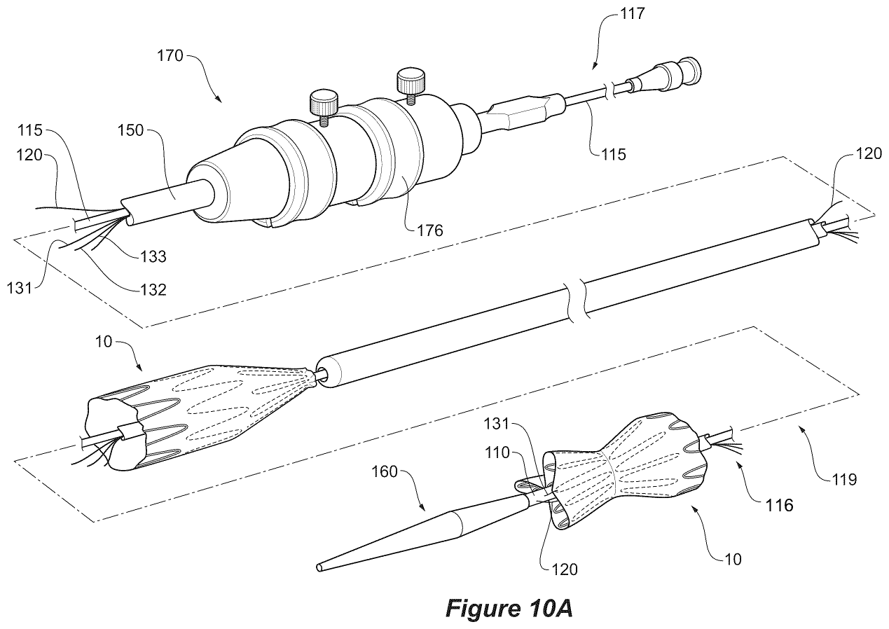

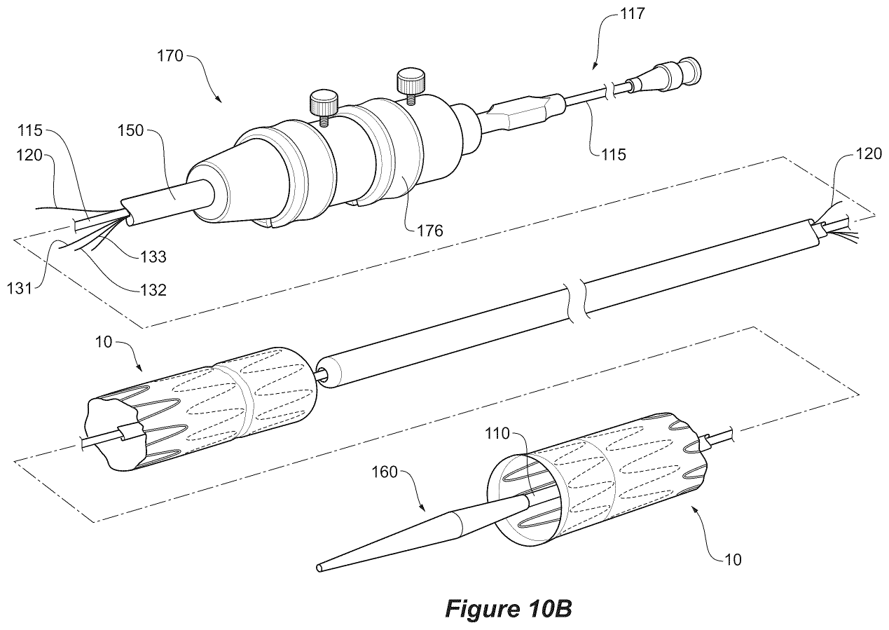

It can be seen from FIG. 1B that a delivery device component of the stent-graft and delivery device assembly may include a sleeve 110, positioned over a guide wire cannula 115 (shown in FIGS. 10A and 10B). The sleeve 110 is constructed from Urethane Radiopaque Tubing (UAT). Other suitable materials and constructions may be used. The sleeve 110, and hence the guide wire cannula 115, passes through the lumen 15.

A graft body apposition release wire 120 releasably securing the graft body 13 with respect to the guide wire cannula 115 along a length of the guide wire cannula 115 is also shown in FIG. 1B. This apposition release wire 120 may repeatedly penetrate the graft body 13 from inside the graft body 13 to outside the graft body 13 around the guide wire cannula 115 and then back from outside the graft body 13 to inside the graft body 13 so as to repeatedly loop over the guide wire cannula 115 along a longitudinal length of the stent-graft 10, thereby securing the stent-graft 10 with respect to the guide wire cannula 115 and against an inner side wall. Elements 124-127 in FIG. 1B show apposition release wire weaving in and out of the graft in the manner described above.

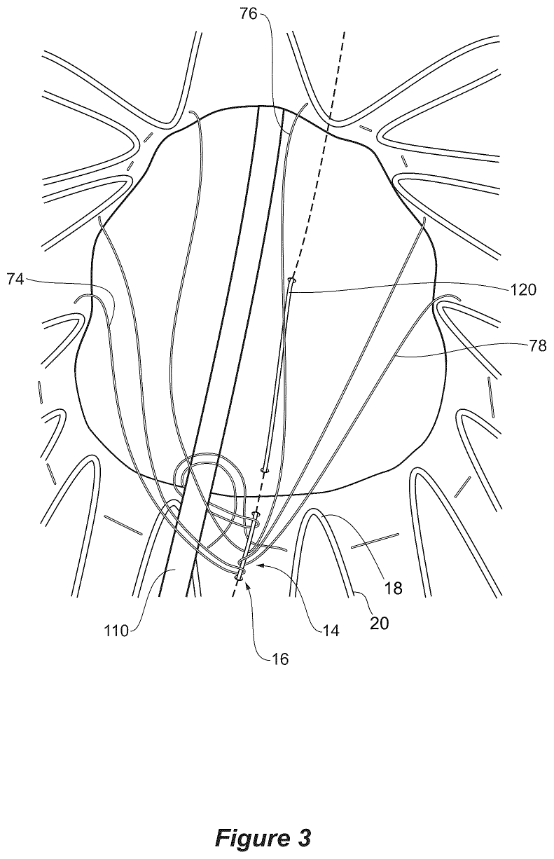

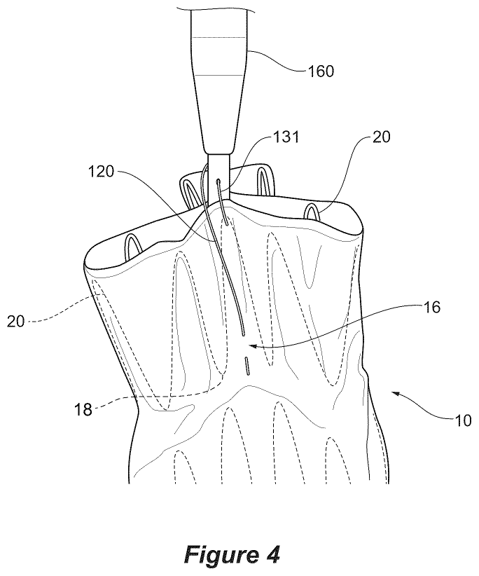

Turning now to FIGS. 1B, 2A, 2B, 4, 5, 6, 10A and 10B, a constraint arrangement for a stent-graft loaded onto a delivery device in combination with a stent-graft 10 according to an embodiment of the disclosure will now be described in further detail. The stent-graft 10 has a graft body 13 that defines an elongate lumen 15 and a positionable region 16. The positionable region 16 is shown in FIGS. 3 and 4. The positionable region 16, in the embodiment shown, is located at a distal end 18 of the proximal most internal stent 20. It is also located to define a 12 o′clock position 23 on the stent graft.

The constraint arrangement comprises: a graft body apposition release wire 120 releasably securing the graft body 13 with respect to the guide wire cannula 115 along a length of the guide wire cannula 115. In the embodiment shown, there is the sleeve 110 over the guide wire cannula 115 so the graft body 13 is also secured around the sleeve 110. The stent-graft may be any type of stent-graft. Particular stent-grafts suitable for use with the present invention are described and disclosed in, for example, U.S. Pat. No. 9,649,188, entitled “Thoracic Aorta Stent Graft,” filed on Feb. 9, 2011; US Publication No. 2018/0303641, filed on Apr. 23, 2018; and US Publication No. 2024/0122695, filed on Oct. 16, 2023, the contents of each of which are incorporated by reference herein in their entirety. Disclosed therein are stent grafts having one or more shaped recess regions having a fenestration and an internal branch extending from the fenestrations. Such stent grafts are particularly suitable for the thoracic aorta or the aortic arch.

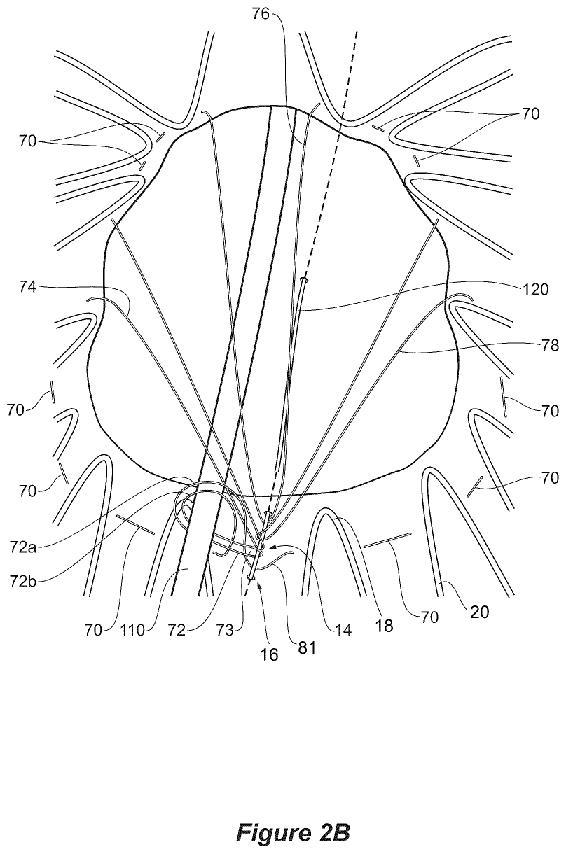

As can be seen clearly by reading FIG. 1B with FIGS. 2A and 2B together, a suture 81 is sewn circumferentially through and around the graft body 13, such that a plurality of external suture portions 80 and a plurality of internal suture portions 70 are formed. A constraint loop 72, having legs 72a and 72b and looped end 73, is formed from a first of the internal suture portions 70 is provided as shown in FIG. 2B. The constraint loop 72 passes around the sleeve 110 and constrains the positionable region 16 in close apposition to the sleeve 110 (and hence the guide wire cannula 115 within the sleeve 110). A plurality of reducing loops 74, 76, 78, formed from others of the internal suture portions 70, extend radially inwards from the graft body 13. The apposition release wire 120 passes through the constraint loop 72 and each of the plurality of reducing loops 74, 76, 78, which reduces the diameter of the stent graft at a position of at least the distal end 18 of the proximal most stent 20 as shown in at least FIG. 1B. In some embodiments, when the stent-graft 10 also includes a side branch located within the most proximal stent, the most proximal ring (e.g., nitinol ring) on the side branch is sitting in an open position and proximal to at least the location where the constraint loop 72 engages the apposition release wire 120. Then the side branch may be compressed (e.g., about a third of the way down), but this has no impact on the side branch as its bracing is made of a flexible material (e.g., nitinol), so it pulls back out into position once the stent-graft is released.

Constraint loops and reducing loops may be placed anywhere along the length of the stent graft as shown in FIG. 1B of US Publication No. 2022/0280280, which disclosure is incorporated by reference herein in its entirety. U.S. Pat. No. 9,855,128, entitled “Introducer for Deploying a Stent Graft in a Curved Lumen and Stent Graft Thererfor,” filed on Oct. 9, 2009, describes and shows reducing loops within the lumen of the stent graft engaging one or more trigger wires, the disclosure of which is incorporated by reference herein in its entirety.

Once the stent graft is introduced and properly placed, retraction of the apposition release wire 120 releases the constraint loop 72 and each of the reducing loops 74, 76, 78 thereby allowing the stent-graft 10 to expand and to release from the guide wire cannula 115, and the sleeve 110.

Again referring to FIGS. 1B and 2B, it can be seen that the apposition release wire 120 penetrates the graft body 13 from outside the graft body 13 to inside the graft body 13 and then back from inside the graft body 13 to outside the graft body 13, thereby providing a substantially linear attachment zone 14 inside the graft body 13 where the constraint loop 72 constrains the positionable region 16 of the stent-graft to the sleeve 110 and the guide wire cannula 115 within the sleeve 110. The apposition release wire 120 includes an apposition release wire proximal end 128. The apposition release wire proximal end 128 sits within the sleeve 110 in a loaded position and the tip (e.g., nose cone) 160 and outside the sleeve 110 in a deployed position.

In FIG. 2B, the constraint loop 72 engages the apposition release wire 120 at a location proximal to a location where the reducing loops (e.g., 74, 76, and 78) engage the apposition release wire 120.

In FIG. 3, an alternative arrangement is shown where the constraint loop 72 engages the apposition release wire 120 at a location distal to a location where the reducing loops (e.g., 74, 76, and 78) engage the apposition release wire 120.

FIG. 4 is a close-up view of a proximal end portion of the stent-graft and delivery device assembly of FIGS. 1B, 2A and 2B. This figure shows a proximal release wire 131 releasably securing a portion of the proximal end of the stent graft to the sleeve 110 and hence the guide wire cannula 115.

Referring again to FIG. 4, it can be seen that the constraint arrangement includes a proximal stent 20 attached to the graft body. The proximal release wire 131 passes through a proximal end of the proximal stent 20. As shown in FIG. 10A and 10B, two other proximal release wires 132, 133 also pass through a proximal end of the proximal stent and hold the proximal stent in a defined position. All three proximal release wires, 131, 132, 133 are shown in FIGS. 10A and 10B and are operably connected to a wire pull mechanism (e.g., a first release knob) 176. U.S. Pat. No. 9,855,128, entitled “Introducer for Deploying a Stent Graft in a Curved Lumen and Stent Graft Therefor,” discloses and describes one suitable configuration for the proximal end of a stent graft, the contents of which are incorporated by reference herein in their entirety. In addition, a distal release wire (not shown) also may be used to hold the distal end of the stent graft. The distal release wire may be directly attached to the distal end of the stent graft by piercing the fabric of the graft at the distal end. Alternatively, the distal release wire may engage a suture (not shown) at the distal end of the stent graft.

Returning to FIGS. 10A and 10B, which show an isometric view of a stent-graft and delivery device assembly according to an embodiment of the disclosure, it can be seen that the delivery device includes a guide wire cannula 115 described above for sliding over a guide wire; sleeve 110 disposed around the guide wire cannula 115; a tip 160 (e.g., a nose cone) at a proximal end 116 of the guide wire cannula 115; a handle 170 at a distal end 117 of the guide wire cannula 115; and a pusher 150 disposed around the guide wire cannula 115 and extending proximally from the handle 170. The delivery device also has a stent-graft receiving zone 119 located between the tip and the pusher, as is shown in FIG. 10A.

Again referring to FIGS. 10A and 10B, a multi-function wire pull mechanism 176 on the handle 170 is provided. The apposition release wire 120 is operably connected to the multi-function wire pull mechanism 176 such that the multi-function wire pull mechanism 176 is actuatable to release the constraint loop 72 and each of the reducing loops 74, 76, 78. This release allows the stent-graft 10 to expand and to release from the guide wire cannula 115.

The multi-function wire pull mechanism 176 on the handle 170 is actuated by an operator, such as a surgeon, pulling in a distal direction. In other embodiments of the disclosure, a rotatable multi-function wire pull mechanism 176 of the type, described in U.S. Pat. No. 8,968,380, entitled “Deployment Handle For An Introducer,” filed on Oct. 9, 2009, and U.S. Pat. No. 10,335,301, entitled “Modular Handle Comprising a Trigger Wire Actuation Mechanism for a Prosthesis Delivery Device,” filed on Aug. 23, 2016, both of which are incorporated by reference herein in their entireties, can be used.

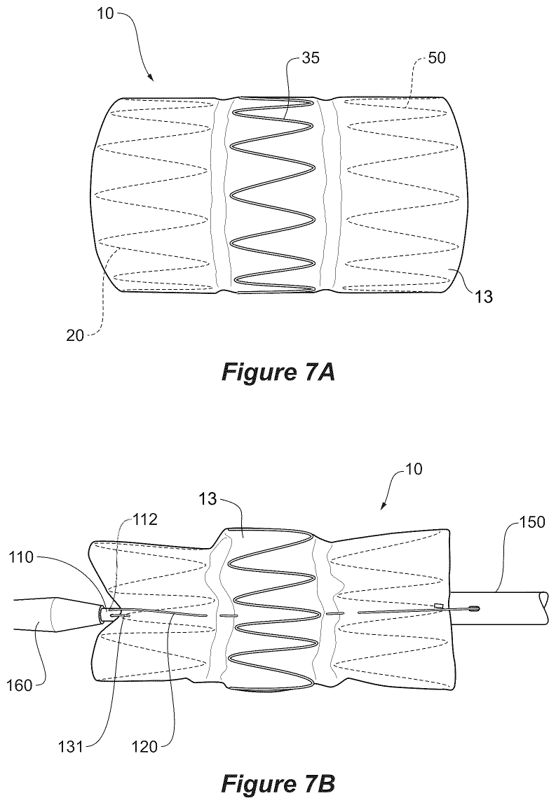

FIG. 7A is a side view of an alternative stent-graft 10 that can be used with embodiment of the disclosure, such as that shown in part in FIG. 7B. This simpler stent-graft 10, when compared to the stent-graft 10 of FIG. 1A for instance, can be used in the ascending aorta for some patients. The stent-graft 10 of FIG. 7A has just one external stent 35 and two internal stents 20, 50. An exemplary device is shown in applicant's U.S. Pat. No. 9,757,263, entitled “Stent Graft and Introducer Assembly,” the contents of which are incorporated by reference herein in their entirety.

FIG. 7B being a side view of an alternative stent-graft and delivery device assembly, shows the stent-graft 10 of FIG. 7A loaded onto a stent-graft receiving zone of a delivery device having a tip 160 (e.g., a nose cone). An apposition release wire 120 is used to releasably secure the graft body 13 with respect to the guide wire cannula 115, within the sleeve 110, along a length of the guide wire cannula 115.

FIG. 8 is a close-up view of a proximal end portion of the stent-graft 10 of FIGS. 7A and 7B. This Figure shows how a proximal release wire 131 releasably attaches a proximal end of the stent-graft to the sleeve 110 and the guide wire cannula 115 within it. An identical, or similar arrangement can be provided at the distal end, with a distal release wire arrangement.

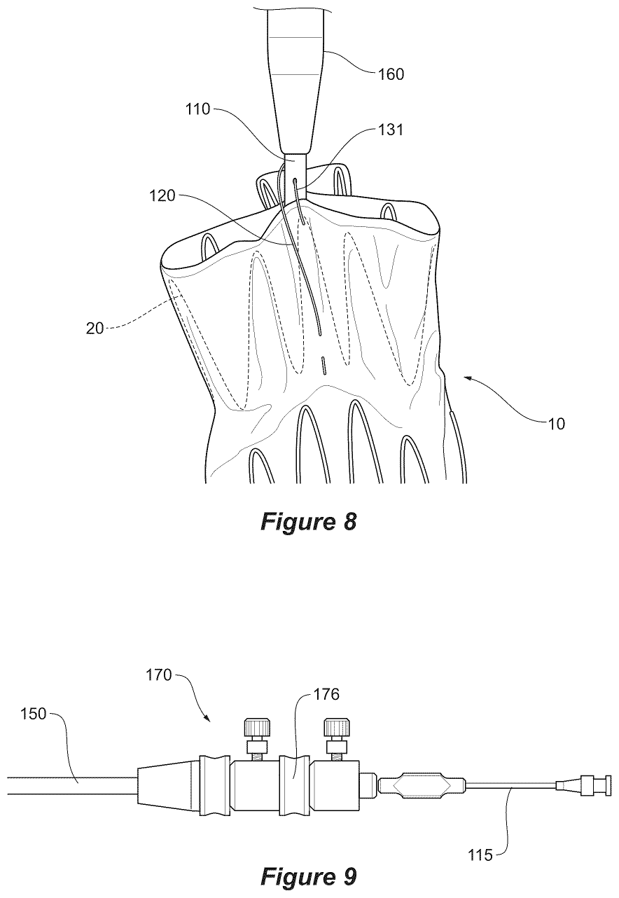

A handle 170 for actuation of the wires is shown in FIG. 9 in a side view of a handle end of the delivery device of FIGS. 7B and 8.

The above-described embodiments of the disclosure reduce risks of entanglement as compared to prior art arrangements. The embodiments also allow for a single actuation releasing the reducing loops and the constraint loop. The embodiments can further release proximal release wire(s) 131, (132, 133) at the same time. The single actuation saves time during an aortic intervention, for instance, reducing risk to the patient who maybe under cardiac control.

The above-described embodiments of the disclosure also are easier to manufacture than prior art stent-graft and delivery device assemblies, saving time and reducing the risks of deployment failures. Also, the combined reducing loops and constraint loop allows the reducing loops and constraint loop to be pulled together and to be released by a single release wire. This saves physician time when a quick release is required under cardiac control. The process of attaching the graft to the delivery device in this manner is also quick and easy. This assists in decreasing aneurysm growth, decreased potential for retrograde tear and decreased reintervention rates, among other things.

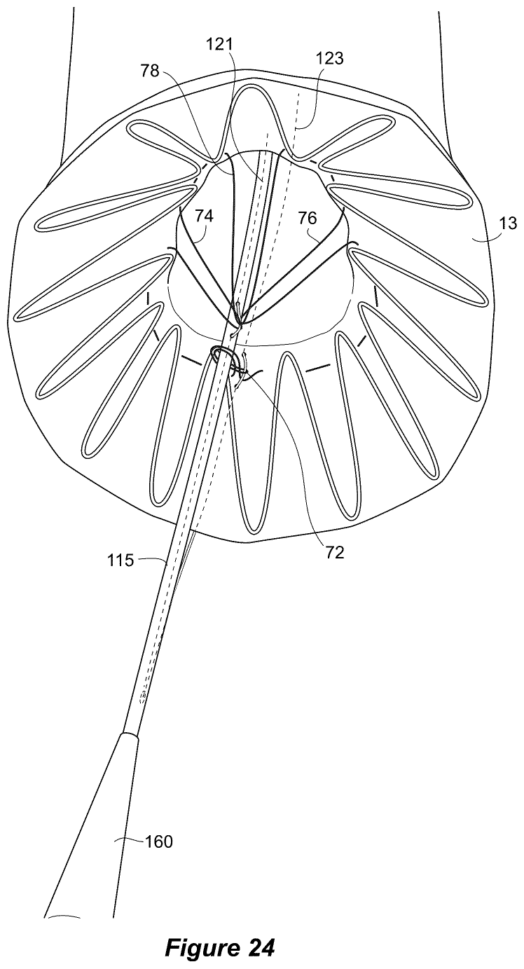

In a first alternative embodiment, as shown in FIG. 21, the reducing loops 74, 76, 78 may be a first suture 83 and the constraint loop 72 may be a separate/second suture 85, each with their own release wire 121, 123. As shown in FIG. 21, a single internal suture portion of the second suture 85 comprises the constraint loop 72 and engages the apposition release wire 123. As further shown, a plurality (e.g., three as shown here) of internal suture portions of the first suture 83 comprises the reducing loops 74, 76, and 78 and engages the release wire 121. The release wire 121 may be external to the guide wire cannula 115, as shown in FIG. 23, or it may extend through the guide wire cannula 115, exit an aperture 129, engage the plurality of the internal suture portions of the reducing loops 74, 76, and 78, and reenter the guide wire cannula 115 at an aperture 139, as shown in FIG. 21.

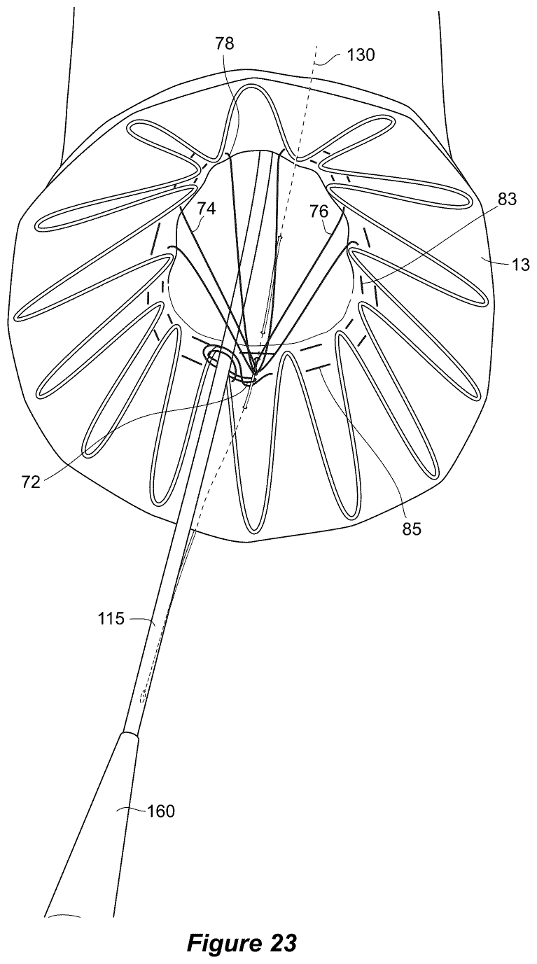

In a second alternative embodiment, as shown in FIGS. 22 and 23, the reducing loops 74, 76, 78 may be a first suture 83 and the constraint loop 72 may be a separate/second suture 85, with a single release wire 130, which operates both as a reducing loop release wire and an apposition release wire. The single release wire 130 may be external to the guide wire cannula 115, as shown in FIG. 23, or it may extend through the guide wire cannula 115, exit an aperture 129, engage the plurality of the internal suture portions of the reducing loops 74, 76, and 78 and the constraint loop 72, and reenter the guide wire cannula 115 at an aperture 139.

In a third alternative embodiment, as shown in FIG. 24, there is a single suture, like what is shown in FIGS. 2A and 2B, but with two release wires, including a reducing loop release wire 121 and an apposition release wire 123. As shown in FIG. 24, the reducing loop release wire 121 engages the plurality of the internal suture portions of the reducing loops 74, 76, and 78, and the apposition release wire 123 engages the constraint loop 72. The reducing loop release wire 121 and the apposition release wire 123 may both be internal to the guide wire cannula 115, may both be external to the guide wire cannula 115, or one of them can be internal to the guide wire cannula 115 and the other one of them can be external to the guide wire cannula 115.

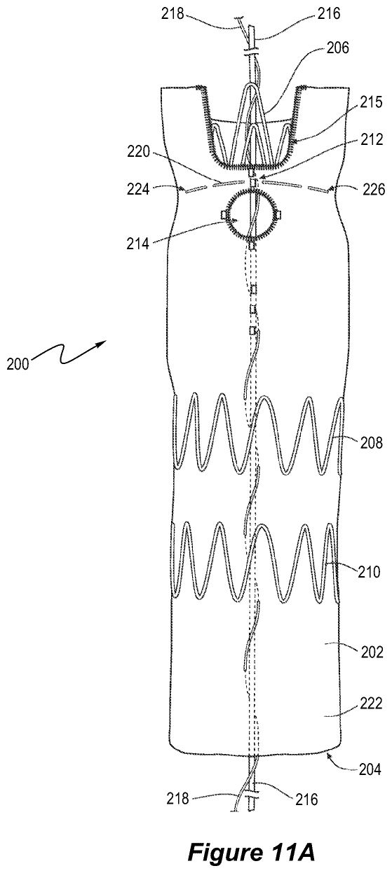

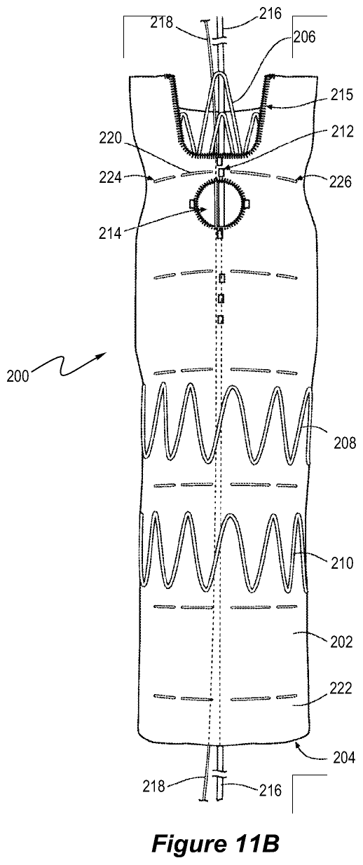

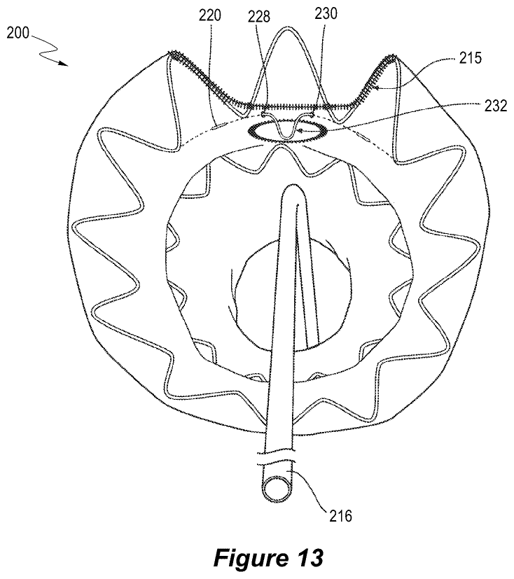

In the embodiments, discussed above, with first and second sutures, the first and second sutures may be longitudinally spaced from one another. For example, the first suture may be proximal of the second suture, or the second suture may be proximal of the first suture. Alternatively, the first and the second sutures may be in the same plane. In the embodiments having first and second sutures, the suture comprising the constraint loop may not extend entirely circumferentially about the stent graft, but only partially, as shown and described in FIGS. 11A, 11B, and 13. Similarly, the suture comprising the reducing loops may not extend entirely circumferentially about the stent graft, but only partially.

The above-described embodiments of the disclosure do not require a special sleeve 110 as only one additional hole in the sleeve 110 is required for the apposition release wire proximal end 128. This hole 112 is shown in FIG. 7B and can be readily made by piercing the apposition release wire proximal end 128 into the sleeve 110 in the position shown in FIG. 7B.

Generally, the more wires that are actuated by a single wire pull mechanism (e.g., a single release knob) on a delivery device, the greater the friction forces that are required to be overcome by the operator of the delivery device. With the arrangements described above, however, it has been found, surprisingly, that a wire pull mechanism (e.g., a first release knob) 176 is able to actuate the three proximal release wires 131, 132, 133 and the apposition release wire 120 simultaneously at a manageable friction force.

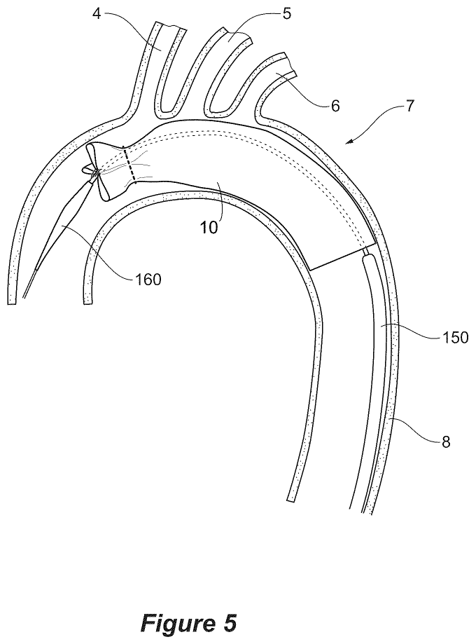

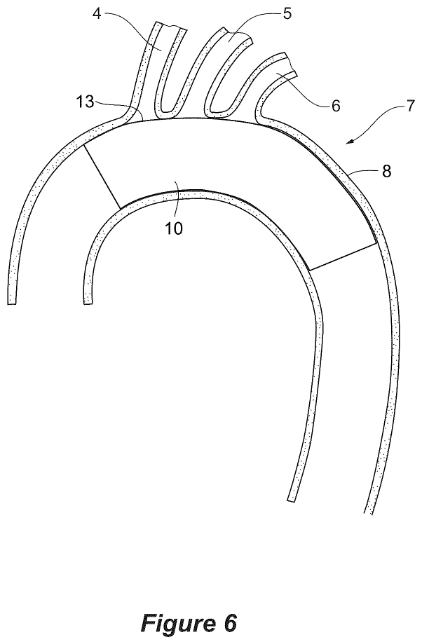

FIGS. 5 and 6 are diagrammatic views showing deployment of the stent-graft and delivery device assembly of FIGS. 1B, 2A and 2B within an aortic arch 7 of a patient. As shown three arteries branch of the aortic arch-the brachiocephalic artery 4, the left common carotid artery 5, and the left subclavian artery 6. FIG. 5 shows the stent-graft 10 on the delivery device ready for deployment in the aortic arch 7. FIG. 6 shows the stent-graft 10 deployed—with no ‘bird beaking’ or gap between the aortic wall 8 and the graft body 13.

A method of making the stent graft includes running a circumferential suture in and out of the graft to create internal loops. The suture crosses the struts of the stent, preferably at the apices on the outside of the graft and preferably between the struts on the inside of the graft. The ends of the suture meet and are knotted at the 12 o'clock position. The apposition release wire, which may be a spiral wire, is run, preferably internally before reaching the distal apices of an internal sealing stent, where it exits the graft below the distal apices, and then re enters the graft, preferably below the distal apices. Using the 12 o'clock suture loop as a suture loop cannula restraint, the loop (which is under the wire), is slipped around the cannula and the wire is run through the loop. In this way, the loop surrounds the cannula and engages the apposition release wire so that the apposition release wire and the cannula are side by side and against the inner wall of the stent graft. Then at least one of the internal suture portions is pulled toward the apposition release wire and the apposition release wire is inserted into the at least one of the internal suture portions. In the embodiments shown in FIGS. 2A, 2B, and 3, the internal suture portions at the 3, 6, and 9 o'clock positions are pulled toward the apposition release wire and the apposition release wire is inserted into each of the internal suture portions at the 3, 6, and 9 o'clock positions. The end of the apposition release wire is then woven from the interior of the graft to the exterior and pulled, which pulls the loops toward the 12 o'clock position. The apposition release wire and cannula are flush with the wall of the graft in a side by side position.

US Publication No. 2022/0280280, filed on Feb. 28, 2022 and published on Sep. 8, 2022, entitled “Constraint Arrangement For a Stent-Graft Loaded Onto a Delivery System” shows a release wire. The contents of the '280 publication are incorporated by reference herein in their entirety.

As described in US Publication No. 2022/0280280, referring to FIG. 11A herein, a constraint arrangement for a stent-graft 200 loaded onto a delivery system according to a first embodiment of the disclosure is shown. The stent-graft has a graft 202 defining an elongate lumen 204, a plurality of stents 206, 208, 210 and at least one positionable region 212 adjacent to a feature, such as a fenestration 214 or a scallop 215. The delivery system has a cannula 216 and a release wire 218.

Now referring to FIGS. 11A, 12A, 12B and 13, it can be seen that the constraint arrangement comprises: a suture 220 extending circumferentially around an outside surface 222 of the graft from a first fixed end 224 fixed to the stent-graft to the positionable region 212, and through the graft 202 at a first constraining perforation 228 (FIG. 13) into the lumen and returning out of the lumen to the outside surface of the graft at a second constraining perforation 230 (FIG. 13) to a second fixed end 226 fixed to the stent-graft 200. A loop 232 of the suture is formed within the lumen. This loop 232 is shown in FIG. 13.

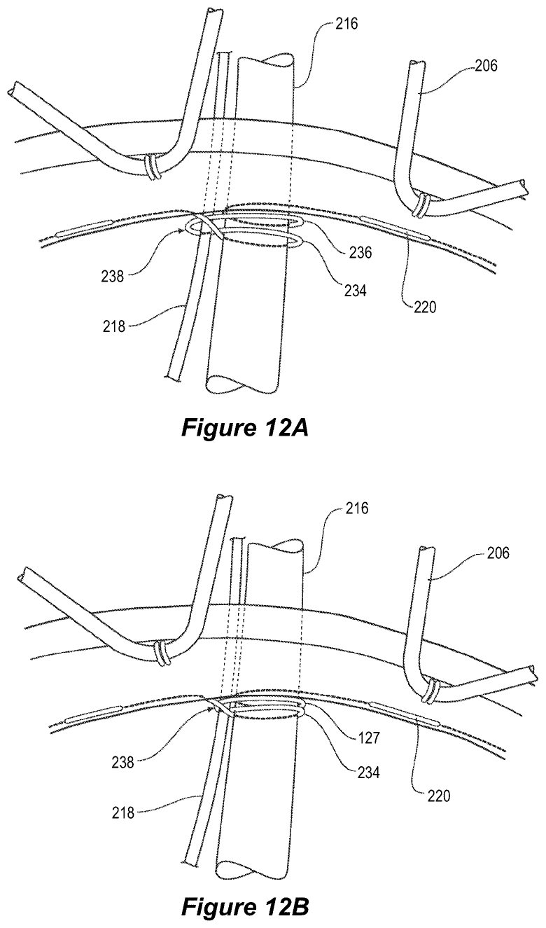

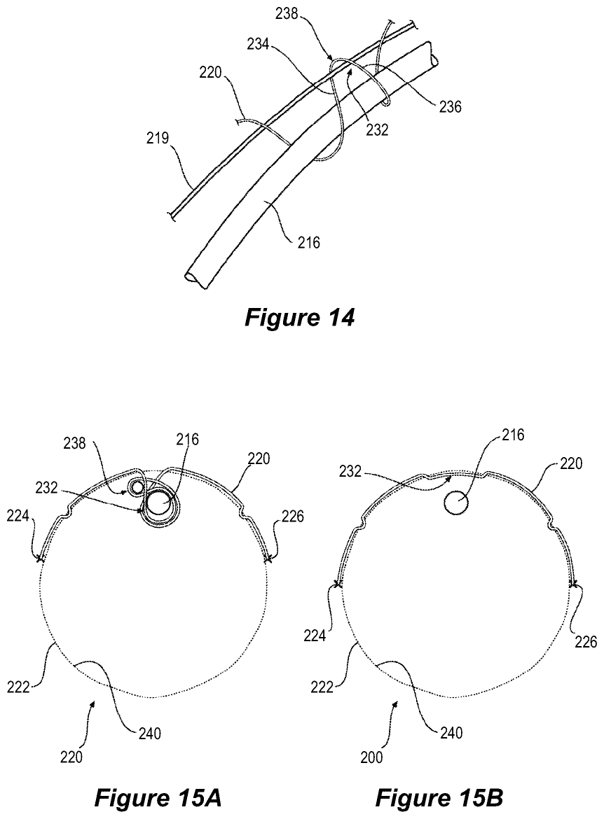

Referring to FIGS. 12A, 12B, 14 and 15A, it can be seen that the loop 232 is wrapped circumferentially around the cannula 216 such that a pair of legs 234, 236 of the loop 232 (most clearly shown in FIG. 14) hold the positionable region 212 (shown in FIGS. 11A and 11B) with respect to the cannula 216. A bight 238 of the loop 232 extends through a gap between the legs 234, 236 and the release wire 218 extends through the bight 238 thereby capturing the bight 238. Thus, the release wire 218 releaseably secures the loop 232 around the cannula 216.

Referring to FIG. 14, read with FIG. 15A, the routing of the suture 220 with respect to the cannula 216 and the release wire 218 is clearly shown before tightening. As can be seen in FIG. 15A, the graft 202 is securely held on the cannula 216 at a specific location or positionable region 212 (shown in FIGS. 11A and 11B). Typically, this positionable region 212 will be at, or adjacent to, an important feature of the stent-graft 200, such as a fenestration 214 or a scallop 215. The graft 202 is named a ‘positionable’ region, because it positionable by the clinician through manipulation of a handle connected to the cannula 216.

The release wire 218 is retractable from the bight 238 of the loop 232 thereby releasing the bight 238 such that the positionable region 212 is no longer held with respect to the cannula 216, as is shown in FIG. 15B.

Using the constraint arrangement for a stent-graft described above, a certain portion of a stent-graft 200 can be controlled more accurately for positioning and alignment purposes. This assists clinicians in various ways. For instance, it assists clinician conducting endovascular procedures for aneurism repair which require the accurate placement of stent-graft features such as a fenestration or scallop 215.

The ability to releasably constrain a required stent-graft feature, such as a fenestration, by utilizing existing components, such as a release or trigger wire is useful and helps keep the overall apparatus that is introduced into the vascular system of patient compact.

Considering FIGS. 15A and 15B together, it can be seen that the constraint arrangement is arranged and constructed such that expansion of the stent-graft 200, when the release wire is in the retracted condition, increases the circumferential length between the fixed end 224 of the suture 220 and the first constraining perforation 228 (as shown on FIG. 13) thereby creating tension in the suture 220. This tension acts to retract the loop 232 towards an inner surface 240 of the graft. This reduces the length of suture within the lumen and also reduces the likelihood of interference with normal blood slow once the procedure of delivering the stent-graft into a patient's anatomy is complete.

The suture 220 penetrates though the graft 202 into and then out of the lumen 204 at least once between the first fixed end 224 and the first constraining perforation 228 as can be seen reading FIGS. 11A and 13 together. With the embodiment shown in FIG. 11A, most of the suture remains on the outside surface 222 of the stent-graft 200, thereby advantageously minimizing the length of suture within the lumen 204.

Again, referring to FIG. 11A, it can be seen that the release wire 218 repeatedly loops over the cannula 216 along a longitudinal length of the stent-graft 200 thereby securing the stent-graft 200 to the cannula 216 at a plurality of spaced-apart locations. With the embodiment illustrated, the release wire repeatedly penetrates the graft from within the lumen to the outside of the lumen and then penetrates the graft from the outside of the lumen to within the lumen. The release wire is disposed helically along the length of the stent-graft, as can be seen in FIG. 11A.

The release wire 218 retains the stent-graft to the cannula, or to a guide wire catheter, and includes a wire wound around the cannula and through the material of the stent-graft in a helical or spiral manner as described above and as illustrated in FIG. 11A. The pitch of the helically wound release wire 218 can be between 20 to 40 mm for example. The release wire 218 extends to a handle for instance, the handle manipulable by a clinician. One suitable release wire is described and shown in US Publication No. 2022/0087812, entitled “Temporary Diameter Reduction Constraint Arrangement for a Stent Graft in Combination With a Stent Graft,” filed Dec. 6, 2021 the entire disclosure of which is incorporated by reference herein it its entirety. Another suitable release wire is described and shown in U.S. Pat. No. 10,188,538 entitled “Hybrid Trigger Wire,” filed on Dec. 30, 2015 the entire disclosure of which is incorporated by reference herein it its entirety.

The suture 220 extends circumferentially around an outside surface 222 of the graft over at least 45 degrees of arc from the fixed end 224 fixed to the stent-graft 200 to the positionable region 212 when the release wire has been retracted and the positionable region 212 is not held with respect to the cannula, such as is shown in FIG. 15B. In the embodiment illustrated in FIGS. 11A, 15A and 15B, the suture extends circumferentially around an outside surface 222 of the graft over at least 75 degrees of arc from the fixed end 224 fixed to the stent-graft to the positionable region, when the release wire has been retracted and the positionable region is not held with respect to the cannula.

In another embodiment of the disclosure, shown in FIG. 11B, a helically wound release wire is not employed. Instead, the release wire is relatively straight and attachment of the stent-graft 200 to the cannula along the length of the stent-graft 200 is achieved by using a plurality of sutures, each suture sewn like the suture 220 as described above.

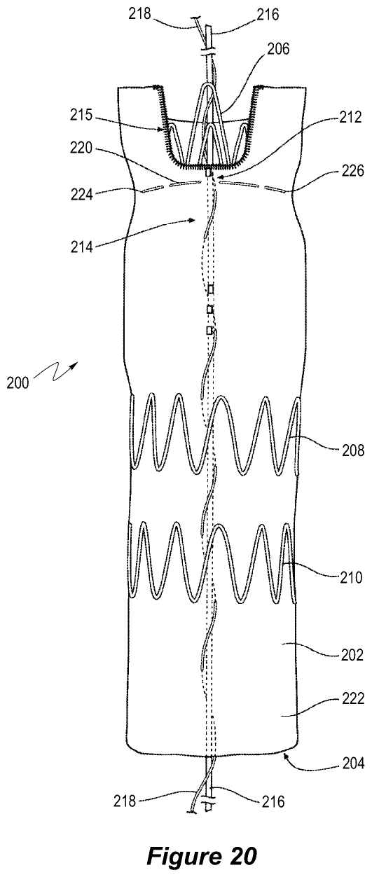

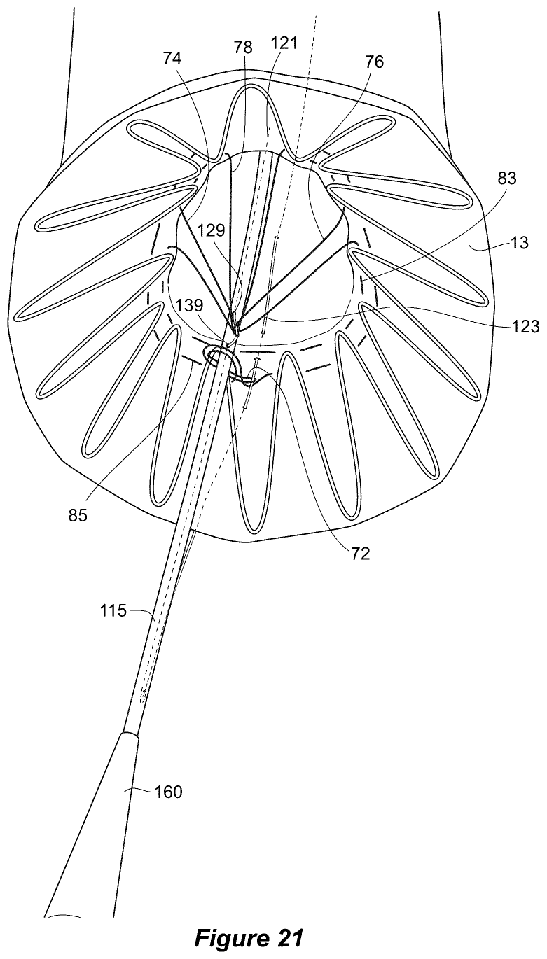

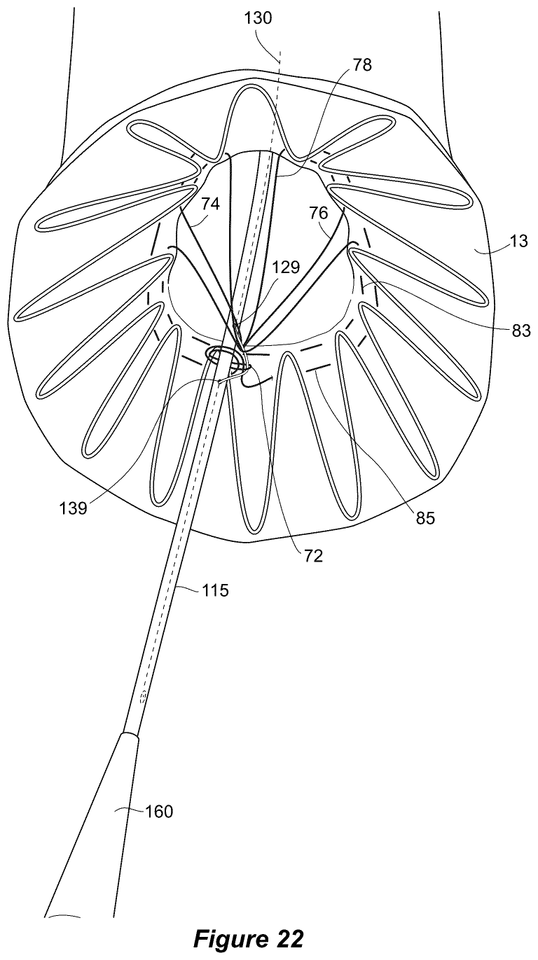

Referring now to FIG. 20, a positionable region is adjacent to a scallop 215, allowing the clinician to precisely control the position of the scallop 215. In other respects, the constraint arrangement is generally the same as that shown in FIGS. 11A, 11B, 12A, 12B, 13, 14, 15A and 15B, where the positionable region is adjacent to a fenestration.

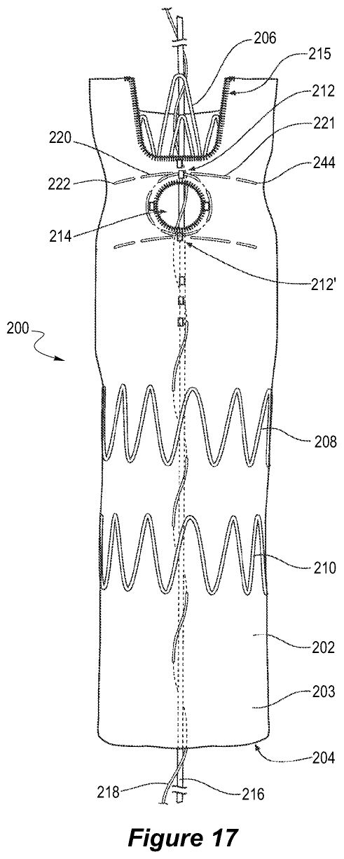

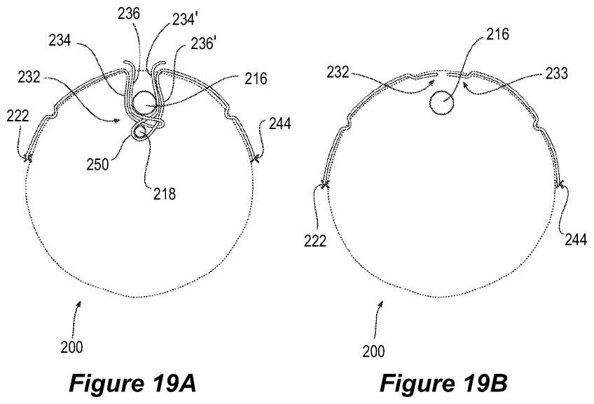

A second embodiment of the disclosure is shown in FIGS. 17 to 19B. In FIG. 17, a constraint arrangement for a stent-graft 200 loaded onto a delivery system is shown. The stent-graft has a graft 200 defining an elongate lumen 204, a plurality of stents 206, 208, 210 and at least one positionable region 212 adjacent to a feature 214. The delivery system has a cannula 216 and a release wire 218. The constraint is similar to that of the constraint arrangement of the first embodiment of the disclosure in many ways, but differs in that two loops of suture are employed, rather than one, for each securement. Specifically, the constraint arrangement comprises a first suture 220 extending circumferentially around an outside surface 203 of the graft from a first fixed end 222 fixed to the stent-graft to the positionable region 212, and through the graft and into the lumen and returning out of the lumen to the outside surface 203 of the graft 202 to a second fixed end fixed to the stent-graft, thereby forming a first loop 232 of the first suture within the lumen. This can be seen in FIGS. 19A and 19B where the first loop 232 is wrapped circumferentially (part circumferentially) around the cannula 216 such that a first pair of legs 234, 236 of the first loop 232 hold the positionable region with respect to the cannula 216.

A second suture 221 also extends circumferentially (part circumferentially) around the outside surface 203 of the graft from a third fixed end 244 fixed to the stent-graft to the positionable region 212, and into the lumen 204 and returning out of the lumen 204 to the outside surface 203 of the graft to a fourth fixed end fixed to the stent-graft, thereby forming a second loop 233 of the second suture within the lumen 204.

A second loop formed by the second suture is wrapped circumferentially around the cannula, such that a second pair of legs 234′, 236′ of the second loop 233 and a first pair of legs 234, 236 of the first loop 232 hold the positionable region 212 with respect to the cannula. A bight 250 of the second loop 23 extends through a gap between the first pair of legs 234, 236 of the first loop 232. The release wire 218 extends through the bight 250 thereby capturing the bight 250 and releaseably secures the first loop 232 and the second loop 220 around the cannula.

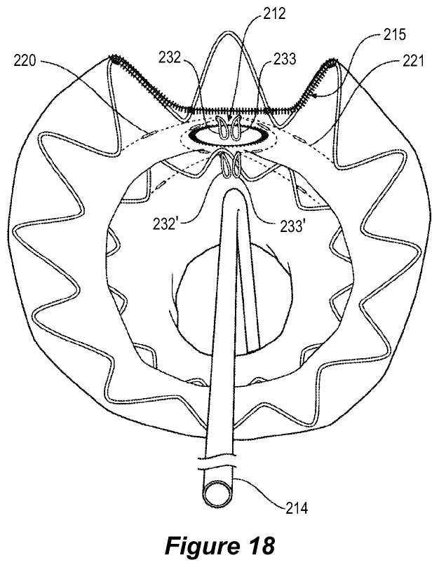

Referring to FIGS. 17 and 18, it can be seen that with this second embodiment there is also a second positionable region 212′ located on the other side of a feature in the form of a fenestration 214. This second positionable region 212′ is held to the cannula 216, providing further control to the clinician. It should be noted however that the double loop arrangement may be employed at just one positionable region 212 if desired. As shown in FIG. 18, third and fourth sutures may be placed below the fenestration (or single suture such as shown in FIG. 11A). As shown third and fourth loops 232′, 233′ prime, like the first and second may be formed.

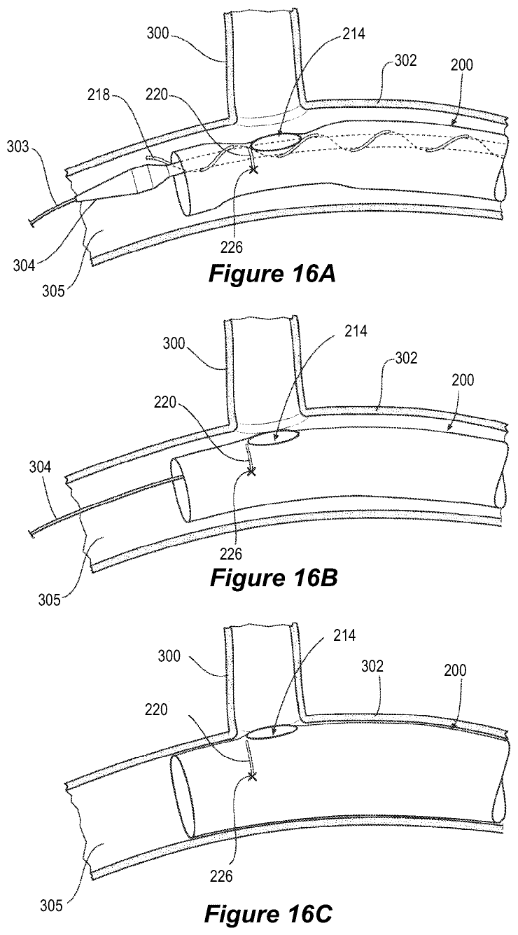

With all of the embodiments described above, the suture loops are greatly reduced (retracted) when the stent-graft is fully released and ballooned, as is shown progressively in FIGS. 16A to 16C. For example, this is achieved in the embodiment illustrated in FIGS. 11A to 15B by firstly weaving the loop material circumferentially around the stent-graft material for about 90 degrees either side of the feature, keeping it firm, but without reducing the stent-graft diameter, or at least not reducing it substantially, and then pulling the loop from the area of the feature that requires constraining. This way, the loop resumes its retracted condition along the circumference of the stent-graft after the stent-graft is released.

Now referring to the embodiment illustrated in FIG. 11A again, a deployment sequence for the constraint arrangement will be described with reference to FIGS. 16A to 16C.

FIG. 16A shows the constraint arrangement within a lumen 2 of a patient in a position where the feature, in this case a fenestration 214, is aligned with a target branch lumen 300 that branches from a main lumen 302. Typically, the constraint arrangement and the stent-graft 200 will have reached this position using the Seldinger technique. This technique involves creating a surgical opening in the vascular system linked to the vessel of interest with a needle and inserting a wire guide into the vessel through a bore of the needle. For example, the femoral artery may be used to access the aorta. The needle can be withdrawn, leaving the wire guide in place. A delivery device is then inserted over the wire guide and into the vessel. The delivery device may be used in conventional fashion to insert into the blood vessel a stent-graft or other prostheses or devices. In FIG. 16A, a guide wire 303 is shown over which a nose cone dilator 304 slides. Adjacent to the nose cone dilator 4 is the stent-graft 200 held to the cannula 216 by a helically wound release wire 218. The constraint arrangement described above with reference to FIGS. 11A, 12A, 12B, 13, 14, 15A and 15B is also partly visible in this Figure, with suture 220 and its first fixed end 224 clearly visible.

While in the position within the main lumen 302, which may be the aorta for instance, the clinician can manipulate the position of the fenestration 214 to align precisely with the opening of the branch lumen 300 (for example a renal artery). The clinician may then cannulate the target vessel with a wire and catheter prior to the final positioning and release of the stent-graft. Once this is complete, the release wire 218 can be retracted, releasing the positionable region 212 adjacent to the fenestration 214 and progressively releasing the stent-graft 200 from the cannula 216 until the position shown in FIG. 16B is attained.

In a final step, the stent-graft 200 is typically further expanded by the deployment of a balloon from within the lumen 204 of the stent-graft 200 until the stent-graft 200 reaches the position shown in FIG. 16C.

Throughout this specification various indications have been given as to the scope of this invention but the invention is not limited to any one of these but may reside in two or more of these combined together. The examples are given for illustration only and not for limitation.

Throughout this specification and the claims that follow unless the context requires otherwise, the words ‘comprise’ and ‘include’ and variations such as ‘comprising’ and ‘including’ will be understood to imply the inclusion of a stated integer or group of integers but not the exclusion of any other integer or group of integers.

1. A constraint arrangement for a stent-graft loaded onto a delivery device in combination with a stent-graft, the stent-graft having: a graft body defining an elongate lumen; and a positionable region, the delivery device having a sleeve passing through the elongate lumen, and the sleeve being disposed around a guide wire cannula, the constraint arrangement comprising:
at least one apposition release wire releasably securing the graft body with respect to the guide wire cannula along a length of the guide wire cannula;
a suture sewn circumferentially through and around the graft body, such that a plurality of external suture portions and a plurality of internal suture portions are formed;
a constraint loop formed from a first of the internal suture portions, the constraint loop passing around the guide wire cannula and constraining the positionable region in close apposition to the sleeve; and
a plurality of reducing loops formed from others of the internal suture portions, the plurality of reducing loops extending radially inwards from the graft body,
wherein the at least one apposition release wire passes through the constraint loop and each of the plurality of reducing loops, and
wherein retraction of the at least one apposition release wire releases the constraint loop and each of the plurality of reducing loops and, thereby allowing the stent-graft to expand and to release from the guide wire cannula.

2. The constraint arrangement as claimed in claim 1, wherein the at least one apposition release wire penetrates the graft body from outside the graft body to inside the graft body and then back from inside the graft body to outside the graft body, thereby providing a linear attachment zone inside the graft body, and wherein the constraint loop constrains the positionable region of the stent-graft to the sleeve.

3. The constraint arrangement as claimed in claim 2, wherein the at least one apposition release wire includes an apposition release wire proximal end, the apposition release wire proximal end within the sleeve in a loaded position and outside the sleeve in a deployed position.

4. The constraint arrangement as claimed in claim 3, wherein the constraint loop engages the apposition release wire at a location proximal to a location where the plurality of reducing loops engages the apposition release wire.

5. The constraint arrangement as claimed in claim 3, wherein the constraint loop engages the apposition release wire at a location distal to a location where the plurality of reducing loops engages the apposition release wire.

6. The constraint arrangement as claimed in claim 1, further comprising at least one proximal release wire, the at least one proximal release wire releasably securing a portion of the positionable region to the guide wire cannula.

7. The constraint arrangement as claimed in claim 6, further comprising a proximal stent attached to the graft body,
wherein the at least one proximal release wire passes through a proximal end of the proximal stent.

8. The constraint arrangement as claimed in claim 1, wherein the apposition release wire repeatedly penetrates the graft body from inside graft body to outside the graft body around the guide wire cannula and then back from outside the graft body to inside the graft body so as to repeatedly loop over the guide wire cannula along a longitudinal length of the stent-graft, thereby securing the stent-graft with respect to the guide wire cannula.

9. A stent-graft and delivery device assembly, the assembly comprising:
a guide wire cannula for sliding over a guide wire;
a sleeve disposed around the guide wire cannula;
a tip at a proximal end of the guide wire cannula;
a handle at a distal end of the guide wire cannula;
a pusher disposed around the guide wire cannula and extending proximally from the handle;
a stent-graft receiving zone located between the tip and the pusher;
a stent-graft having: a graft body defining an elongate lumen; and a positionable region, the stent-graft positioned at the stent-graft receiving zone;
a suture sewn circumferentially through and around the graft body, such that a plurality of external suture portions and a plurality of internal suture portions are formed;
a constraint loop formed from a first of the plurality of internal suture portions, the constraint loop passing around and constraining the guide wire cannula in close apposition to the positionable region;
a plurality of reducing loops formed from others of the plurality of internal suture portions, the plurality of reducing loops extending radially inwards from the graft body;
an apposition release wire releasably securing the graft body to the guide wire cannula along a length of the guide wire cannula, the apposition release wire passing through the constraint loop and each of the plurality of reducing loops; and
a multi-function wire pull mechanism on the handle,
wherein the apposition release wire is operably connected to the multi-function wire pull mechanism such that the multi-function wire pull mechanism is actuatable to release the constraint loop and each of the plurality of reducing loops and, thereby allowing the stent-graft to expand and to release from the guide wire cannula.

10. The stent-graft and delivery device assembly as claimed in claim 9, further comprising:
at least one proximal release wire, the at least one proximal release wire releasably securing a portion of the positionable region to the guide wire cannula,
wherein the multi-function wire pull mechanism is actuatable to release the at least one proximal release wire.

11. The stent-graft and delivery device assembly as claimed in claim 10, wherein the apposition release wire penetrates the graft body from outside the graft body to inside the graft body and then back from inside the graft body to outside the graft body, thereby providing a linear attachment zone inside the graft body where the constraint loop constrains the positionable region of the stent-graft to the sleeve.

12. The stent-graft and delivery device assembly as claimed in claim 11, wherein the apposition release wire includes an apposition release wire proximal end, the apposition release wire proximal end within the guide wire cannula in a loaded position and outside the guide wire cannula in a deployed position.

13. The stent-graft and delivery device assembly as claimed in claim 12, wherein the constraint loop engages the apposition release wire at a location proximal to a location where the plurality of reducing loops engages the apposition release wire.

14. The stent-graft and delivery device assembly as claimed in claim 12, wherein the constraint loop engages the apposition release wire at a location distal to a location where the plurality of reducing loops engages the apposition release wire.
